(19) United States
(12) Patent Application Publication
(10) Pub. No.: US 2025/0176896 A1
(43) Pub. Date: Jun. 5, 2025

(54) INTRACARDIAC MEDICAL DEVICE AND METHOD OF OPERATING SAME

(71) Applicant: BIOTRONIK SE & Co. KG, Berlin (DE)

(72) Inventors: Iris SHELLY, Portland, OR (US); Daniel YOUNG, Portland, OR (US); Kurt SWENSON, Corvallis, OR (US); Madeline Anne MIDGETT, Portland, OR (US); Christopher JONES, Oregon City, OR (US); R. Hollis WHITTINGTON, Portland, OR (US); Hannes KRAETSCHMER, West Linn, OR (US)

(73) Assignee: BIOTRONIK SE & Co. KG, Berlin (DE)

(21) Appl. No.: 18/841,180

(22) PCT Filed: Mar. 3, 2023
(86) PCT No.: PCT/EP2023/055510

§ 371 Date: Aug. 23, 2024

Related U.S. Application Data
(60) Provisional application No. 63/317,206, filed on Mar. 7, 2022.

(30) Foreign Application Priority Data
Mar. 24, 2022 (EP) 22164022.0

(51) Int. Cl. A61B 5/364; A61B 5/00

(52) U.S. Cl. CPC A61B 5/364; A61B 5/686

(57) ABSTRACT

An intracardiac medical device comprising a processing unit and a detector configured to detect time-dependent electrical signals of the heart including ventricular events, for example an intracardiac electrogram, and to transmit the detected electrical signals to the processing unit, wherein the processing unit is configured to classify a current intrinsic ventricular event of the received electrical signals as a normal sense event or a ventricular extrasystole. The invention is further directed to an operation method of such medical device as well as a computer program product and a computer readable data carrier.

### CROSS-REFERENCE TO RELATED APPLICATIONS

This application is the United States National Phase under 35 U.S.C. § 371 of PCT International Patent Application No. PCT/EP2023/055510, filed on Mar. 3, 2023, which claims the benefit of European Patent Application No. 22164022.0, filed on Mar. 24, 2022 and U.S. Provisional Patent Application No. 63/317,206, filed on Mar. 7, 2022, the disclosures of which are hereby incorporated by reference herein in their entireties.

### TECHNICAL FIELD

The present invention is generally directed to an intracardiac medical device and an operation method of such medical device, a respective computer program product and computer readable data carrier.

### BACKGROUND

Intracardiac medical devices such as an Implantable Leadless Pacer (ILP), a (traditional) leaded pacemaker or an Implantable Cardiac Defibrillator (ICD) are generally known.

An implantable intracardiac leadless pacemaker (also known as implantable leadless pacemaker-ILP) is a miniaturized pacemaker which is entirely implanted within a heart's ventricle (V) or atrium (A) of a patient. ILPs are considered the future of cardiac pacing. Due to the highly restricted device size, an ILP has a small battery capacity.

Generally, a pacemaker is a medical device that generates electrical pulses delivered by electrodes connected to or fixed at the pacemaker to cause the heart muscle chambers (i.e., the atria and/or the ventricles) to contract and therefore pump blood. By doing so this device replaces, augments and/or regulates the function of the electrical conduction system of the heart. One purpose of a pacemaker is to maintain an adequate heart rate (cardiac rate), either because the heart's natural pacemaker is not fast enough, or because there is a block in the heart's electrical conduction system. Modern pacemakers are externally programmable and allow a health care professional (HCP) to select the optimal pacing mode(s) for individual patients. The pacing functionality of an ILP (and of a conventional pacemaker) may have a goal of staying synchronized as far as possible with the heart's natural activity.

Additionally, pacemakers are known to use a specific rate for determining the times at which to pace the heart. The specific rate may be realized by counting a clock signal of an interval corresponding to the specific rate. The simplest pacemakers use a fixed, typically programmable, rate which meets the needs of the patient under most circumstances. Using a sensor derived rate that is based on a demand correlated measurement, such as acceleration, is common. For replacing failed AV conduction signals, AV synchronous pacemakers attempt to pace the ventricle at a rate that corresponds with a detected atrial event (e.g., atrial contraction event or atrial depolarization event). It is also possible to deduce a physiologically missing electrical heart rate signal from other systems in the body (e.g., the brain and baroreceptors) from other sensors. An inherent rate used by all modern pacemakers is the intrinsic rate of the heart.

ILPs largely aim to provide support as has been delivered for bradycardia management in traditional, pocket based leaded pacemakers through devices sized at ˜10% of the total volume of legacy formats. A VDD operation mode may be realized using such ILP based on detected electrical signals of a detector which determines time-dependent electrical signals of the heart including ventricular events (e.g., ventricular contraction events or ventricular depolarization events). While the miniaturization permits the placement of leadless implants within the blood volume of a patient's heart and provides reduced risks for regurgitation and infection through the elimination of leaded interfacing with the myocardium, the approach eliminates the faculties for directly measuring cardiac signaling emergent from a multitude of heart chambers and instead uses an implant that resides wholly within a single heart chamber, e.g., within a ventricle. There is therefore a need to collect heart chamber information readily available to leaded systems “at a distance” in leadless embodiments to facilitate synchronous output from the device. This capability demands that the affiliate mode support architecture be one that offers means to effectively manage behaviors subject to a reduced reliability in the quality of, e.g., atrial, input signaling and event detection.

An intracardiac medical device such as a (traditional) leaded pacemaker, or an ICD may sense the patient heart's activity similar to the above-mentioned ILP. An ICD provides defibrillation functions to treat life-threatening arrhythmias whereas the intracardiac heart rhythm monitor collects data with regard to the patient's heart. Analogously to the above-described ILP such devices may be accommodated at least for a pre-defined time within a heart's ventricle (V) or atrium (A) of a patient and therefore may face the same challenges as the ILP discussed above.

A ventricular extrasystole (VES), also known as a premature ventricular contraction (PVC), is an early contraction of the ventricles, with the electrical signal originating in the ventricles themselves rather than being conducted from the atria. In a patient with normal AV conduction experiencing a single VES, the VES is usually preceded by a short interval from the prior ventricular event, and it is followed by a long compensatory pause interval before the next ventricular event.

Traditional dual chamber pacemakers with an atrial lead can classify VES events based on the presence or absence of an atrial event preceding the intrinsic ventricular event. However, for an intracardiac medical device, in particular without a detector directly in atrium, atrial sensing must occur based on an atrial signal as seen by the device from, e.g., within the ventricle. As such, atrial sensing may be less reliable and more prone to undersensing than in a traditional dual chamber pacemaker. Accordingly, recognition of VES events using the classic way for an intracardiac medical device cannot be as robust as for a conventional dual chamber pacemaker.

While an individual VES event is not typically of clinical significance, larger numbers of VES events may become clinically significant, making it beneficial for an implanted cardiac device to provide a statistic regarding the prevalence of VES events. Additionally, because the intrinsic cardiac timing is typically modified following a VES, an intracardiac leadless pacemaker may be better able to maintain synchrony with the intrinsic activity of the patient's heart by modifying its pace timing following a VES. To make this possible, VES classification of a ventricular event must happen in real time.

Accordingly, there is the need for an intracardiac medical device and a respective operation method that allows reliable identification of VES events in real time.

The present disclosure is directed toward overcoming one or more of the above-mentioned problems, though not necessarily limited to embodiments that do.

### SUMMARY

At least the above problem is solved by a method of operating an intracardiac medical device with the features of claim 1, by a respective intracardiac medical device with the features of claim 8, by a computer program product with the features of claim 14, and by a computer readable data carrier with the features of claim 15. The computer program product may be a software routine and/or related hardware support means with the intracardiac medical device.

In particular, at least the above problem is solved by a method of operating an intracardiac medical device within a patient's heart, wherein the medical device comprises a processing unit and a detector, wherein the detector detects time-dependent electrical signals of the heart including ventricular events, for example, an intracardiac electrogram (IEGM), and transmits the detected electrical signals to the processing unit, wherein the processing unit classifies a current intrinsic ventricular event of the received electrical signals as a ventricular extrasystole (VES) if a temporal distance (or time interval or elapsed time) of the current intrinsic ventricular event from a directly preceding ventricular event meets at least one pre-determined prematurity criterion and otherwise classifies the current intrinsic ventricular event as a non-VES ventricular event. For example, an R-R peak temporal distance of a detected R peak of the current intrinsic ventricular event from a detected R peak of a directly preceding ventricular event is assessed with regard to the at least one pre-determined prematurity criterion. In the above example the R-peak of the current intrinsic ventricular event and the R-peak of the directly preceding ventricular event is taken to determine the temporal distance of the current intrinsic ventricular event and the directly preceding ventricular event. Other signal features of the electrical signals (e.g., an IEGM) may be used for determining the temporal distance, as well, for example, the Q or S components of the ventricular depolarization.

The term “directly preceding ventricular event” means that the ventricular event is from the directly preceding cardiac cycle of the patient's heart.

The intracardiac medical device may be, for example, an ILP, an ICD or a single lead pacemaker.

With regard to the present invention, the processing unit is generally regarded as a functional unit of the pacemaker, that interprets and executes instructions comprising an instruction control unit and an arithmetic and logic unit. The processing unit may comprise or be a microprocessor, a controller, a digital signal processor (DSP), an application specific integrated circuit (ASIC), a field-programmable gate array (FPGA), discrete logic circuitry or any combination thereof. Alternatively, or additionally, the processing unit may be realized using integrated dedicated hardware logic circuits, in particular, in the case of an ILP due to the small size and extreme power limitation. The processing unit may comprise a state determining module, wherein with regard to an ILP as a medical device the state determining module is configured to dynamically select one of a plurality of states and to use the selected state to determine the ventricular pacing time and/or rate information to meet the patient's actual therapeutic needs. Examples of different states with regard to an ILP are explained below in more detail.

The processing unit processes electrical signal data of the patient's heart received from the detector. In particular, the detector is configured to detect the time-dependent electrical depolarization and repolarization field signals such as an intracardiac electrogram (IEGM). These signals comprise signals caused by the depolarization and repolarization of the atria (in the following, the intrinsic atrial event) and electrical signals caused by the depolarization and repolarization of the ventricles (in the following, the intrinsic ventricular event). The intrinsic atrial event may be a far field signal (e.g., an electrical signal) if the medical device, for example, the ILP, is located within one ventricle. The detector may preprocess these data, for example, digitize the signals, filter them and/or amplify them.

The processing unit to which the electrical signals of the detector are transmitted perceives intrinsic ventricular events and, if applicable, intrinsic atrial events from the electrical signals, for example, the intrinsic atrial event from the P wave and the intrinsic ventricular event from the QRS complex, in particular from the R peak of the QRS complex. Since the far-field electrically measured P wave is much smaller in amplitude compared to near-field ventricle signals, a higher amplification may be used in a time period of the signal in which the P wave is expected than in the QRS and T-wave time intervals. Alternatively, the electrical signal input provided by the sensor may be split into two channels internal to the processing unit-one for the atrium and one for the ventricle. Each channel has its own amplification and filtering schemes to respectively detect an intrinsic atrial event and an intrinsic ventricular event.

The processing unit may further comprise a counter and a clock. The counter may be used to count clock signals of the clock. The counter may be started at each sensed atrial or ventricular depolarization and count the number of clock cycles until the next atrial or ventricular depolarization occurs or ventricular pacing is provided by the pacing signal generator to determine a temporal distance of the current intrinsic ventricular event from the directly preceding ventricular event, for example, a R-R peak temporal distance.

The pacemaker may comprise a data memory which may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The data memory saves the above and below mentioned thresholds and conditions. They are required by the processing unit during processing the above and below explained steps. The data memory may further save measured values of temporal distances with regard to the current intrinsic ventricular event and the directly preceding ventricular event such as R-R peak temporal distances and, if applicable, an actual duration of one cardiac cycle or the corresponding actual cardiac rate and, if applicable, the expected duration of one cardiac cycle.

According to the above-described operating method of the medical device the processing unit classifies a current intrinsic ventricular event of the received electrical signals as a ventricular extrasystole (VES) if the temporal distance of the current intrinsic ventricular event from a directly preceding ventricular event meets at least one pre-determined prematurity criterion. If none of at least one prematurity criterion is not met, the current intrinsic ventricular event is classified as a “normal” ventricular event, in the following referred to as a “non-VES event” (Vs). Prior to the VES classification the processing unit may determine, whether the current ventricular event is an intrinsic ventricular event or a ventricular pacing event, for example, by the timing of the event and/or the signal form.

The current intrinsic ventricular event is the most recently detected intrinsic ventricular event. The directly preceding ventricular event may be an intrinsic ventricular event or a ventricular pacing event which was detected by the detector or provided by the device within the directly previous cardiac cycle.

In one embodiment, a prematurity criterion is met if the temporal distance of the current intrinsic ventricular event from the directly preceding ventricular event is significantly shorter than an actual or expected duration of one cardiac cycle and/or is equal to or less than a pre-defined cardiac cycle prematurity duration threshold.

For example, the temporal distance of the current intrinsic ventricular event from the directly preceding ventricular event is equal to or shorter than a prematurity threshold determined from the expected duration of one cardiac cycle in one of several possible ways:
The prematurity threshold is determined as a fixed or programmable multiplicative factor (e.g., between 0.5 and 0.95) times the expected duration of one cardiac cycle or
the prematurity threshold is determined as a fixed or programmable duration (between 50 and 500 ms) shorter than the expected duration of one cardiac cycle.

Therein, in one embodiment, the expected duration of one cardiac cycle is the duration of the most recent cardiac cycle without any VES event or is an average of the durations of a pre-defined number of most recent cardiac cycles without any VES event, e.g., the average of the duration of most recent non-VES cardiac cycles may be maintained as the average of a rolling interval buffer between 1 and 64 cycles, or as an exponential moving average with weights ranging from 1 (no history, most recent interval only) to 1/64 (long history, where each cycle contributes only 1/64 towards the average). In at least one embodiment, the expected duration above and below is an actual duration.

In one embodiment, the cardiac cycle prematurity duration threshold may be a fixed or programmable duration threshold (e.g., a single value from 300 ms to 1000 ms).

The above classification of a ventricular event as a VES or non-VES event allows immediate (real time) classification of VES events, without waiting to measure the interval after the potential VES event, allows the pacemaker to modify its behavior for that following cycle, for example, to accommodate the expected compensatory pause in intrinsic activity immediately after a VES, which improves the ability to maintain far-field atrial detection and AV synchrony after a VES.

In one embodiment, application of the pre-determined prematurity criterion to the temporal distance of the current intrinsic ventricular event from the directly preceding ventricular event is suspended and the current intrinsic ventricular event is classified as non-VES ventricular event if an atrial event is detected by the processing unit from the received electrical signals after the time point of the R peak of the directly preceding ventricular event and/or if the atrial event is detected in a temporal distance from the current intrinsic ventricular event that is shorter than the duration of the actual cardiac cycle and/or shorter than the pre-defined normal cardiac cycle length. This means that an intrinsic atrial event preceding the intrinsic ventricular event in question (current intrinsic ventricular event) is used to disqualify it from classification as a VES. In other words, a valid sensed intrinsic atrial event between the preceding ventricular event and the intrinsic ventricular event in question would prevent classification of the ventricular event as a VES. Accordingly, in this embodiment, a VES must be preceded by a non-VES cardiac cycle that does not meet the at least one prematurity criterion, and must not be preceded by a valid sensed atrial event. Accordingly, classification of a VES by a medical device is provided based on a combination of at least one prematurity criterion and (if available) atrial sensing information. Hence, VES classification is performed based on ventricular event prematurity and, if available, the presence of atrial information allows an intracardiac leadless pacemaker implanted in the ventricle to more accurately classify VES events even during periods of intermittent atrial sensing.

In one embodiment, application of the pre-determined prematurity criterion to the temporal distance of the current intrinsic ventricular event from the directly preceding ventricular event is at least temporarily suspended or disabled and the current intrinsic ventricular event is classified by the processing unit as non-VES ventricular event at least during a time period in which a state determining module of the processing unit uses at least one pre-determined state of a plurality of states, wherein such state may be adopted by the processing unit of an ILP, for example, to determine the ventricular pacing time and/or rate information to meet the patient's actual therapeutic needs. This allows VES classifications to be performed only when relevant (for example, only when attempting atrial sensing so that atrial information may be available and ILP activity is more likely to be synchronized with intrinsic cardiac activity). This means that VES classifications may be differently enabled and disabled in different programmed modes (e.g., a VDD mode) or in different states within a mode (e.g., an atrial tracking state within a VDD mode, or a motion sensor driven state within a VDD mode).

One example state that may be controlled by above-mentioned state determining module of the processing unit of an ILP to determine ventricular pacing time and/or rate information is VDD mode with atrial tracking. This mode assumes that the patient has some form of intrinsic AV conduction disorder, either complete or intermittent. It also assumes that the sinus node is generally competent, and the VDD mode attempts to track the sinus rate and to provide AV synchronization. The AV delay may be determined based on the actual cardiac rate (which corresponds to the actual duration of one cardiac cycle) and may be set to the usual time difference between the intrinsic atrial and ventricular contraction or depolarization. From the actual cardiac rate, the current AV delay may be determined by a known calculation or using a look-up-table contained in the data memory. The current AV delay changes with the actual cardiac rate. Alternatively, a fixed, pre-defined AV delay may be used.

The “ventricular pacing time and/or rate information” includes any timing information a pacing signal generator of an ILP that is connected to the processing unit needs to produce the ventricular pacing signal at the correct time, i.e., according to the treatment plan. The ventricular pacing time and/or rate information may be, in part determined or dictated by, an AV delay and/or a cardiac rate and/or the duration of a cardiac cycle. In one embodiment, the ventricular pacing time and/or rate information may comprise a hysteresis time to be added to the AV delay, so that pacing is slightly delayed, to allow for more time to sense an intrinsic ventricular event.

Based on the pacing control signal, the pacing signal generator of an ILP produces the electrical pacing signal(s) to transfer it to the electrodes which apply the signal(s) to the heart's tissue adjacent to the electrode. The pacing signals are pulses that begin at a desired time point and have a desired intensity and duration. Further, the pulse waveform may be varied. Pacing control signaling is used by the processing unit to instruct the pacing signal generator on specifics associated with the duration, timing, and amplitude of the administered therapy output pulses. In particular, the ventricular pacing control signal contains ventricular pacing time and/or rate information. In one embodiment, the pacing signal is not determined (i.e., inhibited) or not transferred to the electrodes if an intrinsic ventricular event is detected within a predefined time period (e.g., the AV delay) after the detected intrinsic atrial event in the state operating according to the VDD mode.

In a different state, a so-called intermediate state, the ventricular pacing time and/or rate information may be determined from ramping an initial cardiac rate to a pre-defined pacing rate, e.g., the resting rate of the patient. The initial cardiac rate may be the actual cardiac rate of the time point at which the state determining module switched to the intermediate state. The intermediate state may be left to another state as soon as the pre-defined pacing rate is reached. Such state transitions aim to ensure the administration of baseline bradycardia mitigation therapy while also striving to adapt to measurable patient needs: minimize the introduction of noticeable symptoms thought the avoidance of abrupt rate changes; and return device/patient interactions to optimal conditions with minimal delay. In another intermediate state the processing unit may attempt AV resynchronization using tracking of intrinsic atrial and ventricular events to synchronize pacing with intrinsic activity of the heart to return to a state using VDD mode with atrial tracking, for example.

In one embodiment, the state determining module is configured such that it selects a state that uses the signals received from at least one second detector comprised by the ILP, for example, an accelerometer, a vibration sensor, an impedance sensor, an acoustic sensor (including ultrasound) and/or other mechanical, electric and/or magnetic sensor that is capable of detecting time-dependent activity of the patient, for the determination of the ventricular pacing time and/or rate information depending on whether the usage of the respective second detector is allowed, for example, by the HCP. Such state may be a VVI-R mode. This state has the advantage that the HCP may adapt the operation of the cardiac pacemaker better to the patient's needs if the patient is active. Accordingly, in this state the patient activity is tracked and the ILP and its processing unit may be structured to turn off any capacity to monitor atrial input signaling. Thereby the longevity of the power source (battery) is increased. A state operating according to the known VVI mode using a pre-defined pacing rate for this mode may be provided by the processing unit and monitored by the state determining module, as well.

For example, VES classification may be separately enabled during a state having AV synchronous tracking condition, e.g., in a state using VDD mode, and may be disabled in an intermediate state where AV resynchronization is attempted and/or in a state in which patient activity is tracked using above-mentioned second sensor (and which operates without AV synchrony) and/or in a state using VVI mode. In this embodiment, VES classification would only occur for states in which this feature is enabled.

The ability to enable and disable VES classifications in different states allows VES classifications to be performed only when relevant (for example, only when attempting atrial sensing so that atrial information may be available and, in case of an ILP, pacemaker activity is more likely to be synchronized with intrinsic cardiac activity).

Intervals that include a VES cycle are not indicative of the true intrinsic heart rate, so these intervals are excluded from use by the medical device to update the above VES prematurity criteria. As such, this prematurity-based VES classification may be vulnerable to a ‘VES lock-in’ behavior, where a pattern of repeated VES classifications caused by a long prematurity threshold can prevent updates to that prematurity threshold, perpetuating a pattern of VES classifications that are unlikely to be indicative of true ventricular extrasystoles. For example, in the case of a sudden increase in a conducted intrinsic rate without atrial sensing information, rapid conducted ventricular events may be consistently misclassified as VES events based on a long prematurity threshold. In this condition, the VES classifications would continually prevent the prematurity threshold from being updated to a shorter value consistent with the new intrinsic rate, thus allowing the condition to persist indefinitely.

Accordingly, in one embodiment, application of the pre-determined prematurity criterion to the temporal distance of the current intrinsic ventricular event from the directly preceding ventricular event is at least temporarily suspended or disabled and the current intrinsic ventricular event is classified by the processing unit as non-VES ventricular event at least during a time period in which a pre-defined VES suspension criterion is met, for example, using a VES counter (VES up/down counter), where each classified VES event increments the VES counter and each non-VES ventricular event (including ventricular pacing event) decrements it. At least one VES suspension criterion of the following criteria may be, for example, applied.
The VES suspension criterion is met if a VES counter value is greater than or equal to a pre-defined suspension threshold (e.g., a first counter maximum value and a suspension threshold is programmable, for example, from 1 to 32) or
the VES suspension criterion is met if the VES counter value is greater than or equal to X out of Y ventricular events from recent cycles classified as VES events (e.g., X and Y from 1 to 32) or
the VES suspension criterion is met if a pre-defined number N of consecutive VES events occurred (e.g., N from 1 to 32).

In one embodiment, application of the pre-determined prematurity criterion to the temporal distance of the current intrinsic ventricular event from the directly preceding ventricular event is re-enabled if a pre-defined VES re-enablement criterion is met, for example,
The VES re-enablement criterion is met if VES counter value is less than a pre-defined re-enable threshold (a second counter maximum value and a re-enable threshold is programmable, for example, from 1 to 32) or
the VES re-enablement criterion is met if ventricular events of less than X out of Y recent cardiac cycles classified as VES events (e.g., X and Y from 1 to 32) or
the VES re-enablement criterion is met if a pre-defined VES suspension timeout (e.g., 1 to 60 seconds) elapses or
the VES re-enablement criterion is met if a pre-defined VES suspension number of cardiac cycles (e.g., 1 to 32 ventricular events) have occurred.

The ability to temporarily suspend and re-enable VES classifications allows recognition of a ‘VES lock-in’ situation with frequent ILP classified VES events. These situations may benefit from a temporary suspension of VES classifications to allow use of the measured ventricular intervals to update ILP internal values related to pace timing and/or the prematurity threshold used for future VES classifications.

In one embodiment, the above method is realized as a computer program (to be executed at or within the intracardiac medical device, in particular utilizing its processing unit) which is a combination of above and below specified (computer) instructions and data definitions that enable computer hardware to perform computational or control functions and/or operations, or which is a syntactic unit that conforms to the rules of a particular programming language and that is composed of declarations and statements or instructions needed for an above and below specified function, task, or problem solution.

Furthermore, a computer program product is disclosed comprising instructions which, when executed by a processing unit of an intracardiac medical device, cause the processing unit to perform the steps of the above defined method. Accordingly, a computer readable data carrier storing such computer program product is described.

At least the above problem is further solved by an intracardiac medical device comprising a processing unit and a detector configured to detect time-dependent electrical signals of the heart including ventricular events, for example, an intracardiac electrogram, and to transmit the detected electrical signals to the processing unit, wherein the processing unit is configured to classify a current intrinsic ventricular event of the received electrical signals as a ventricular extrasystole (VES) if a temporal distance of the current intrinsic ventricular event from a directly preceding ventricular event meets at least one pre-determined prematurity criterion and to classify the current intrinsic ventricular event otherwise as a non-VES ventricular event.

The embodiments described for the method of operating a medical device apply to the above medical device, as well, causing the analogous advantages. It is referred to above embodiments of the method in this regard.

In one embodiment of the medical device, the processing unit is configured such that a prematurity criterion is met if the temporal distance of the current intrinsic ventricular event from the directly preceding ventricular event is significantly shorter than an expected duration of one cardiac cycle and/or is equal to or less than a pre-defined cardiac cycle prematurity duration threshold, wherein, for example, the expected duration of one cardiac cycle is the duration of the most recent cardiac cycle without any VES or is an average of the durations of a pre-defined number of most recent cardiac cycles without any VES.

In one embodiment of the medical device, the processing unit is configured such that an application of the pre-determined prematurity criterion to the temporal distance of the current intrinsic ventricular event from the directly preceding ventricular event is suspended and the current intrinsic ventricular event is classified as non-VES ventricular event if an atrial event is detected by the processing unit from the received electrical signals after the time point of the R peak of the directly preceding ventricular event and/or if the atrial event is detected in a temporal distance from the current intrinsic ventricular event that is shorter than the duration of the actual cardiac cycle and/or shorter than the pre-defined cardiac cycle prematurity duration threshold.

In one embodiment of the medical device, the processing unit is configured such that the application of the pre-determined prematurity criterion to the temporal distance of the current intrinsic ventricular event from the directly preceding ventricular event is at least temporarily suspended or disabled and the current intrinsic ventricular event is classified by the processing unit as non-VES ventricular event at least during a time period in which a state determining module of the processing unit uses at least one pre-determined state of a plurality of states.

In one embodiment of the medical device, the processing unit is configured such that the application of the pre-determined prematurity criterion to the temporal distance of the current intrinsic ventricular event from the directly preceding ventricular event is at least temporarily suspended or disabled and the current intrinsic ventricular event is classified by the processing unit as non-VES ventricular event at least during a time period in which a pre-defined VES suspension criterion is met, for example, by using a VES counter.

In one embodiment of the medical device, the processing unit is configured such that an application of the pre-determined prematurity criterion to the temporal distance of the current intrinsic ventricular event from the directly preceding ventricular event is re-enabled if a pre-defined VES re-enablement criterion is met.

Together, the above method and medical device allows reporting of a VES prevalence statistic to HCP. For that, the medical device may comprise further modules such as a communication unit for communication with a remote computer. The communication unit may exchange messages with an external (at least partially extracorporeally) remote computer, for example, in one single direction or bidirectionally. The communication may be provided wirelessly via the patient's body, preferably acoustic, conducted and/or magnetically coupled, and/or the air using electromagnetic waves, for example, MICS-band, Bluetooth, WLAN, ZigBee, NFC, Wibree or WiMAX in the radio frequency region, or IrDA or free-space optical communication (FSO) in the infrared or optical frequency region or by wire (electrical and/or optical communication). The remote computer is a functional unit that can perform substantial computations, including numerous arithmetic operations and logic operations without human intervention, such as, for example, a personal mobile device (PMD), a desktop computer, a server computer, clusters/warehouse scale computer or embedded system. The medical device's units and components may be contained within a hermetically sealed housing.

In one embodiment, the intracardiac medical device may comprise a power supply such as a battery to supply electrical power to the modules/units/components of the medical device. The power supply therefore is electrically connected to the respective modules/units/components of the medical device.

In one embodiment, in case the medical device is an ILP, the medical device may comprise electrodes for application of an electrical pacing signal provided by the pacing signal generator. The electrodes are electrically connected to the pacing signal generator via a header of the ILP. One electrode may be located at a distal end of the ILP, close to a fixation member by which the ILP is fixed in the tissue of the patient's heart, for example, against or within the tissue of a ventricle. A second electrode may be located at the proximal end of the ILP or a part of the ILP housing that may, for example, serve as counter electrode. Further, in one embodiment, the electrodes may additionally be adapted to detect the intrinsic ventricular event or the intrinsic atrial event in each case over time by picking up electrical potentials. The electrodes may thereby be part of the detector of the medical device.

These features can also allow the intracardiac leadless pacemaker to adjust pace timing following a VES to encourage maintaining AV synchrony.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawing, wherein

FIG. 1 shows an embodiment of an intracardiac medical device, namely, an ILP, within a cross section of a patient's heart,

FIG. 2 depicts a functional block diagram of the ILP shown in FIG. 1,

FIG. 3 shows an enlarged side view of the ILP of FIG. 1, and

FIG. 4 shows a flow chart of an embodiment of the operation method of the ILP of FIG. 1.

### DETAILED DESCRIPTION

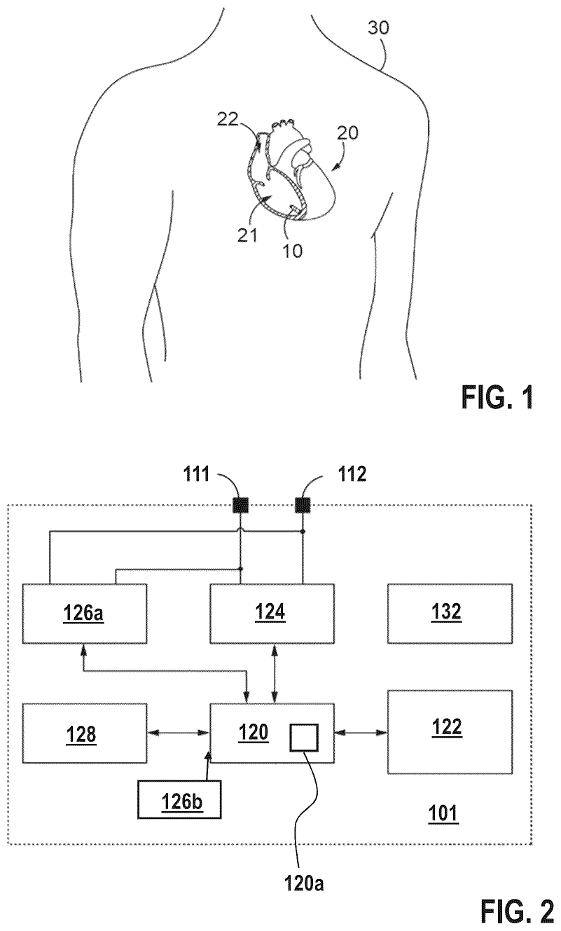

In the following, the present invention is described with regard to an ILP as one embodiment. FIG. 1 shows such leadless ventricular pacemaker (ILP) 10 implanted within the heart 20 of a patient 30. ILP 10 may be configured to be implanted within the right ventricle 21 of the heart 20 and pace this ventricle, sense intrinsic ventricular depolarizations and depolarizations of the atria (e.g., the right atrium 22) and inhibit ventricular pacing in response to detected ventricular depolarization. A programmer (not shown) may be used to program ILP 10 and retrieve data from ILP 10 via a wireless communication connection for which examples are explained above. The ILP 10 is one example of an intracardiac medical device. Other embodiments of an intracardiac medical device such as a cardiac monitor are possible, as well.

FIG. 2 shows a functional block diagram of the ILP 10 configured for implantation within right ventricle 21 (FIG. 1). The ILP 10 comprises a processing unit 120 with a clock, at least one counter for the clock signals and a data memory 122, a pacing signal generator 124, a detector unit comprising a first detector 126a and at least one second detector 126b, a communication unit 128, and a power source 132. The power source 132 may be electrically connected to one or more of the other components 120, 122, 124, 126a, 126b, 128 (not shown in FIG. 2) and may include a battery, e.g., a rechargeable or non-rechargeable battery. The power source provides electrical energy to all units and components of the ILP 10 (not explicitly shown in the figure), in particular to all units mentioned above and is therefore electrically connected to these units and components. Similar or identical units and functionality may also be included in the ILP 10. Units of the medical device of present disclosure may include any discrete and/or integrated electronic circuit components that implement analog and/or digital circuits capable of producing the functions attributed to the units herein. For example, the units may include analog circuits, e.g., amplification circuits, filtering circuits, and/or other signal conditioning circuits. The units may also include digital circuits, e.g., combinational or sequential logic circuits, memory devices, etc. The units may further be realized using integrated dedicated hardware logic circuits. The data memory 122 may include any volatile, non-volatile, magnetic, or electrical media mentioned above. Furthermore, the processing unit 120 may include instructions that, when executed by one or more processing circuits, cause the units to perform various functions attributed to these units herein. The functions attributed to the units herein may be embodied as one or more processors, hardware, firmware, software, or any combination thereof. Depiction of different features as units is intended to highlight different functional aspects and does not necessarily imply that such units must be realized by separate hardware or software components. Rather, functionality associated with one or more units may be performed by separate hardware or software components or integrated within common or separate hardware or software components. Data memory 122 may store computer-readable instructions that, when executed by processing unit 120, cause processing unit 120 to perform the various functions attributed to processing unit 120 herein. Further, data memory 122 may store parameters for these functions, e.g., pacing signal parameters, conditions and thresholds described above and below. The pacing instructions and pacing signal parameters, conditions and thresholds may be updated by the programmer using the communication unit 128. The communication unit 128 may comprise an antenna, coil, and/or a transceiver.

The processing unit 120 may communicate with pacing signal generator 124 and detectors 126a, b thereby transmitting signals. Pacing signal generator 124 and the first detector 126a are electrically coupled to electrodes 111, 112 of the ILP 10. The first detector 126a is configured to monitor signals from electrodes 111, 112 to monitor electrical activity of heart 20. The second detector 126b may be realized as a motion sensor, for example, an accelerometer or any other motion sensor described above. However, an accelerometer-based motion sensor does not necessarily require any connection with the electrodes 111, 112. The motion sensor collects time-dependent motion signals as known from an accelerometer and transmits these signals to the processing unit 120, wherein the signals may be pre-processed similarly to the signals of the first detector 126a as described below.

Pacing signal generator 124 is configured to deliver electrical stimulation signals to ventricle 21 via electrodes 111, 112.

The first detector 126a may further include circuits that acquire time-dependent electrical signals (e.g., electric depolarization and repolarization signals) from the heart including intrinsic cardiac electrical activity, such ventricular events and, if applicable, intrinsic atrial events. The first detector 126a may filter, amplify, and/or digitize the acquired electrical signals of the heart chambers contractions and include support for splitting inputs from the electrodes into multiple channels for subsequent handling by 120. Processing unit 120 may receive the time-dependent signals generated by first detector 126a including ventricular events and, if applicable, intrinsic atrial events.

Processing unit 120 may assess the electrical signals, identify ventricular events and, if applicable, the intrinsic atrial events received from the first detector 126a and is configured to determine the (time-dependent) actual cardiac rate corresponding to the actual duration of one cardiac cycle, calculate an expected duration of one cardiac cycle, as well as classify the intrinsic ventricular event with regard to ventricular extrasystole as described below in detail. Processing unit 120 may further control pacing signal generator 124 to deliver electrical stimulation therapy according to one or more therapy programs including pacing parameters, which may be stored in data memory 122.

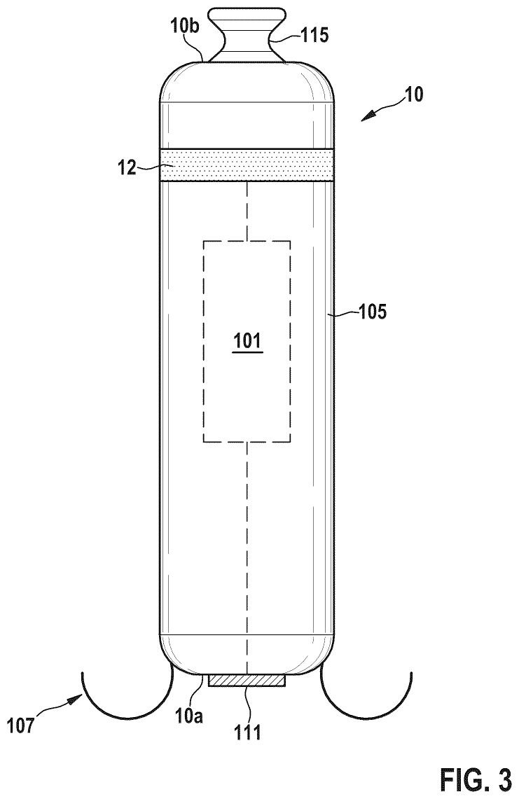

ILP 10 may include a housing, anchoring fixation features (tines) 107, and the electrodes 111, 112 at a first end 10a and near a second end 10b of the ILP 10. Electronic components 101 (see FIGS. 2, 3) as shown in FIG. 2 with regard to their functionality are electrically connected to the electrodes 111, 112. The housing may have a pill-shaped cylindrical form factor in some examples. The anchoring fixation features 107 are configured to connect ILP 10 to heart 20. Anchoring fixation features 107 may be fabricated from a shape memory material, such as Nitinol. In some examples, anchoring fixation features may connect ILP 10 to heart 20 within one of the chambers of heart 20. For example, as illustrated and described herein with respect to FIG. 1, anchoring fixation features 107 may be configured to anchor ILP 10 to heart 20 within right ventricle 21. Although ILP 10 includes a plurality of anchoring fixation features/elements 107 that are configured to stably engage ILP 10 to cardiac tissue in the right ventricle, it is contemplated that a pacemaker according to the present disclosure may be engaged with cardiac tissue in other chambers of a patient's heart 20 using other types of anchoring fixation features. The housing further comprises a catheter engagement hitch 115 at the second end 10b of the ILP 10.

ILP 10 may include two electrodes 111, 112, although more than two electrodes may be included on a pacemaker in other examples. As depicted in FIG. 3 electrodes 111, 112 may be spaced apart a sufficient distance to be able to detect various electrical signals generated by the heart 20, such as P-waves generated by atria and QRS complex generated by ventricles. The housing houses electronic components of ILP 10. Electronic components may include any discrete and/or integrated electronic circuit components that implement analog and/or digital circuits capable of producing the functions attributed to ILP 10 described above.

The communication unit 128 may enable ILP 10 to communicate with other electronic devices, such as a programmer or other external patient monitor, for example, to communicate VES statistic to the HCP. In some examples, the housing may house a coil and/or an antenna for wireless communication. The housing may also include the power source 132.

The processing unit 120 may be adapted to control pacing of the right ventricle 21 in a state using the known VDD mode based on the intrinsic atrial event containing atrial contractions and, if applicable, the intrinsic ventricular event containing ventricular contractions. The counter of the processing unit 120 used to time the AV delay (for providing the ventricular pace signal) may also be used to measure intrinsic AV delays. The VDD pacing mode may be R-Sync in the ILP 10. This means that every cycle is synchronized by every used ventricular event (intrinsic ventricular contraction or ventricular pacing). It is also an atrial tracking mode. This means that every sensed atrial contraction can shift the timing. In other words, VDD is both R-Sync and P-Sync. The timing of the next potential ventricular pacing signal is scheduled based on the most recent ventricular event and a targeted pacing interval (determined from a target cardiac rate). Sensed atrial contractions “re-schedule” the next pacing signal by starting an AV interval. The actual cardiac rate and thereby the actual duration of one cardiac cycle is determined from the intrinsic atrial event and, if applicable, from the intrinsic ventricular event and/or from the paced output from the pacemaker (ILP 10). To control the pacing, the processing unit comprises a state determining module 120a (see FIG. 2) which represents a part of the algorithm that is used to dynamically switch between a state operating according to the VDD mode and several other states to determine the ventricular pacing time and/or rate information dependent on the present condition of the patient and key parameters pre-defined by the HCP.

As indicated above, the state determining module 120a may operate in the VDD mode which means to have the ventricular event (either an intrinsic ventricular contraction or a ventricular pacing signal if the intrinsic AV conduction is inadequate) track the intrinsic atrial contractions. In other states the ventricular pacing time and/or rate information may be determined using a VVI-behavior or a VVI-R behavior in the at least one second state as explained below in more detail. The VVI behavior comprises a pacing that does not consider atrial activity but is based on a pre-defined pacing rate. The VVI-R behavior provides a pacing rate dependent on the signal of the second detector representing the actual activity of the patient. In one embodiment, in a state using a VVI or VVI-R mode atrial tracking is not provided. There may be a further state in which the processing unit may re-synchronize the intrinsic atrial and ventricular events and the pacing to return to the state using the VDD mode. There may be an additional non-atrial-tracking transition state in which is based on a pre-defined pacing rate, e.g., the resting rate. For example, a pacing rate is ramped from an initial (actual) cardiac rate to the pre-defined pacing rate. The resting rate may be pre-defined for the specific patient, either directly or indirectly, by input from the HCP and stored in the data memory. The VES classification of detected ventricular events may be enabled or disabled if the state determining module 120a switches between the different states. For example, VES classification may be enabled in the atrial-tracking state but disabled in the transition state, in which the pacing rate is ramped to the pre-defined pacing rate, in the state using VVI or VVI-R behaviour and in the state in which the ventricular pacing is being resynchronized to the intrinsic atrial activity. Additionally, VES classification may be enabled or disabled for different timer modes, for example, enabled when programmed by a HCP to a VDD mode but disabled when programmed to a VVI or VVI-R mode.

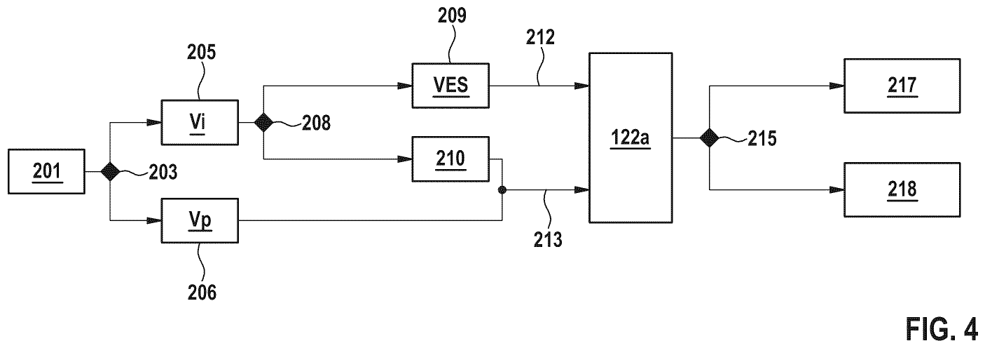

For example, in the state using the VDD mode VES classification is enabled and operates as follows. The operation of VES classification is explained with regard to FIG. 4.

The first detector 126a continuously monitors by the electrodes 111, 112, the electrical activity of the heart 20 and transmits the detected electrical signals (potentially the filtered, amplified and/or digitized electrical signals) to the processing unit 120, for example, in the form of an IEGM. The processing unit 120 analyses these electrical signals with regard to the presence of a ventricular event. If a ventricular event is detected in the current cardiac cycle (see step 201 in FIG. 4), it is analyzed (see step 203 in FIG. 4) to determine whether the detected ventricular event is an intrinsic ventricular event Vi (see box 205 in FIG. 4) or a pacing ventricular event Vp (see box 206 in FIG. 4). These ventricular events may be distinguished with regard to their timing and/or their signal form. As the processing unit 120 controls the pacing it knows when a pacing signal was applied and may compare the received ventricular event signal with its expectation of a ventricular pacing event Vp. An intrinsic ventricular event is assumed if no pacing was provided within the corresponding cardiac cycle.

If VES classification is not suspended (see below), in the next step 208 the processing unit 120 now assesses the intrinsic ventricular event Vi of the current cardiac cycle whether it is a VES event or it is a non-VES event (Vs). The intrinsic ventricular event Vi is regarded a VES event (see box 209 in FIG. 4) if a temporal distance of the current intrinsic ventricular event from a directly preceding ventricular event meets at least one above-explained pre-determined prematurity criterion and otherwise the intrinsic ventricular event is regarded as a non-VES event (Vs) (see box 210 in FIG. 4). For example, an R-R peak temporal distance of a detected R peak of the current intrinsic ventricular event from a detected R peak or similar feature of a directly preceding ventricular event is assessed whether it meets the at least one pre-determined prematurity criterion. For example, the R-R peak temporal distance is equal to or shorter the prematurity threshold which is, for example, 0.8 times the expected duration of one cardiac cycle.

In one embodiment, in step 218 it may be taken into account whether the first detector 126a and/or the processing unit 120 has/have detected an atrial event prior the current intrinsic ventricular event and after the previous ventricular event. If this is the case, in step 218 the intrinsic ventricular event is classified as non-VES event (Vs) (box 210). Accordingly, the prematurity criteria are not applied to the temporal distance of the current intrinsic ventricular event from the directly preceding ventricular event in this case. If there is no atrial event within this temporal distance, the prematurity criteria are applied.

The classification is finished after the intrinsic ventricular event is classified as VES event (box 209) or non-VES event (Vs) (box 210) and the information with regard to the property of the current intrinsic ventricular event may be used by the processing unit 120 for adaption of the pacing time for the present and/or next cardiac cycle. Additionally, cardiac cycles in which a VES event is detected may not be taken into account for determination of a current cardiac rate or, analogously, of a current duration of one cardiac cycle. Further, the detected VES events may be saved in the data memory 122 for statistics reason and/or communicated to an external device using the communication unit 128.

Additionally, as shown in FIG. 4, in one embodiment the data memory 122 may comprise a VES counter 122a. The counter may be counted up by one, for example, if a VES event 209 was detected (see arrow 212). The counter may be counted down by one, for example, if a non-VES event 210 (Vs) or a ventricular pacing event 206 (Vp) was detected (see arrow 213).

In order to avoid any perpetuating pattern of VES classifications that are unlikely to be indicative of true ventricular extrasystoles the VES counter 122a is compared with regard to a pre-defined suspension threshold which has, for example, the value 30 in step 215. If the counted number of the VES counter 122a is equal to or greater than 30, the VES classification is suspended (see step 217 in FIG. 4). Otherwise, the VES classification stays active. If the VES classification is suspended and in step 215 the comparison of the VES counter 122a with a re-enable threshold (value may be, e.g., 15) reveals that the VES counter value is lower than this threshold, the VES classification is re-enabled (see step 218 in FIG. 4). Otherwise, the VES classification stays suspended.

The above described classification of intrinsic ventricular events as VES events and non-VES events (Vs), as well as the possibility to suspend and re-enable this classification under certain circumstances allows to more accurately track the cardiac rate of the patient and to compensate an extrasystole during pacing. Further, the information on the intrinsic ventricular events may be used to adapt the therapy, for example, by provision of a different pacing device or different pacing parameters, if a pathologic situation is observed with regard to ventricular extrasystoles.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A method of operating an intracardiac medical device within a patient's heart, wherein the medical device comprises a processing unit and a detector, wherein the detector detects time-dependent electrical signals of the heart including ventricular events, for example an intracardiac electrogram, and transmits the detected electrical signals to the processing unit, wherein the processing unit classifies a current intrinsic ventricular event of the received electrical signals as a normal ventricular sense or a ventricular extrasystole if a temporal distance of the current intrinsic ventricular event from a directly preceding ventricular event meets at least one pre-determined prematurity criterion and otherwise as a non-VES ventricular event.

2. The method of claim 1, wherein a prematurity criterion is met if the temporal distance of the current intrinsic ventricular event from the directly preceding ventricular event is significantly shorter than an expected duration of one cardiac cycle and/or is equal to or less than a pre-defined cardiac cycle prematurity duration threshold.

3. The method of claim 2, wherein the expected duration of one cardiac cycle is the duration of the most recent cardiac cycle without any VES or is an average of the durations of a pre-defined number of most recent cardiac cycles without any VES.

4. The method of claim 1, wherein application of the pre-determined prematurity criterion to the temporal distance of the current intrinsic ventricular event from the directly preceding ventricular event is suspended and the current intrinsic ventricular event is classified as non-VES ventricular event if an atrial event is detected by the processing unit from the received electrical signals after the directly preceding ventricular event and/or if the atrial event is detected in a temporal distance from the current intrinsic ventricular event that is shorter than the duration of the actual cardiac cycle and/or shorter than the pre-defined cardiac cycle prematurity duration threshold.

5. The method of claim 1, wherein application of the pre-determined prematurity criterion to the temporal distance of the current intrinsic ventricular event from the directly preceding ventricular event is at least temporarily suspended or disabled and the current intrinsic ventricular event is classified by the processing unit as non-VES ventricular event at least during a time period in which a state determining module of the processing unit uses at least one pre-determined state of a plurality of states.

6. The method of claim 1, wherein application of the pre-determined prematurity criterion to the temporal distance of the current intrinsic ventricular event from the directly preceding ventricular event is at least temporarily suspended or disabled and the current intrinsic ventricular event is classified by the processing unit as non-VES ventricular event at least during a time period in which a pre-defined VES suspension criterion is met, for example by using a VES counter.

7. The method of claim 1, wherein application of the pre-determined prematurity criterion to the temporal distance of the current intrinsic ventricular event from the directly preceding ventricular event is re-enabled if a pre-defined VES re-enablement criterion is met.

8. An intracardiac medical device comprising a processing unit and a detector configured to detect time-dependent electrical signals of the heart including ventricular events, for example an intracardiac electrogram, and to transmit the detected electrical signals to the processing unit, wherein the processing unit is configured to classify a current intrinsic ventricular event of the received electrical signals as a ventricular extrasystole if a temporal distance of the current intrinsic ventricular event from a directly preceding ventricular event meets at least one pre-determined prematurity criterion and to classify the current intrinsic ventricular event otherwise as a non-VES ventricular event.

9. The medical device of claim 8, wherein the processing unit is configured such that a prematurity criterion is met if the temporal distance of the current intrinsic ventricular event from the directly preceding ventricular event is significantly shorter than an expected duration of one cardiac cycle and/or is equal to or less than a pre-defined cardiac cycle prematurity duration threshold, wherein, for example, the expected duration of one cardiac cycle is the duration of the most recent cardiac cycle without any VES or is an average of the durations of a pre-defined number of most recent cardiac cycles without any VES.

10. The medical device of claim 8, wherein the processing unit is configured such that an application of the pre-determined prematurity criterion to the temporal distance of the current intrinsic ventricular event from the directly preceding ventricular event is suspended and the current intrinsic ventricular event is classified as non-VES ventricular event if an atrial event is detected by the processing unit from the received electrical signals after the directly preceding ventricular event and/or if the atrial event is detected in a temporal distance from the current intrinsic ventricular event that is shorter than the duration of the actual cardiac cycle and/or shorter than the pre-defined cardiac cycle prematurity duration threshold.

11. The medical device of claim 8, wherein the processing unit is configured such that the application of the pre-determined prematurity criterion to the temporal distance of the current intrinsic ventricular event from the directly preceding ventricular event is at least temporarily suspended or disabled and the current intrinsic ventricular event is classified by the processing unit as non-VES ventricular event at least during a time period in which a state determining module of the processing unit uses at least one pre-determined state of a plurality of states.

12. The medical device of claim 8, wherein the processing unit is configured such that the application of the pre-determined prematurity criterion to the temporal distance of the current intrinsic ventricular event from the directly preceding ventricular event is at least temporarily suspended or disabled and the current intrinsic ventricular event is classified by the processing unit as non-VES ventricular event at least during a time period in which a pre-defined VES suspension criterion is met, for example by using a VES counter.

13. The medical device of claim 8, wherein the processing unit is configured such that an application of the pre-determined prematurity criterion to the temporal distance of the current intrinsic ventricular event from the directly preceding ventricular event is re-enabled if a pre-defined VES re-enablement criterion is met.

14. A computer program product comprising instructions which, when executed by the processing unit, cause the processing unit to perform the steps of the method according to claim 1.

15. Computer readable data carrier storing a computer program product according to claim 14.
